SEQUENCE LISTING (5 sequences)

SEQ ID NO: 1 - DNA, 1005 bp, Bradyrhizobium japonicum
atgcaggacacgaaattcaaagtcgcggtcgttcaggctgcgccggtattcatggatgcgccagcttccgtggccaaggcgattggtttcattgcggaggcgggtgcagcgggggcgaagctgctggcgttcccggaggtctggattccgggctatccttggtggctttggctcgggacgccggcttggggaatgcagtttgtaccccgctatcacgccaattcgctgcgtgctgatgggcccgacatcctcgcactgtgtgcggccgccgccgaagcgaaaatcaatgtggtgatgggcttctccgaaatcgacggaggaacgctctacctaagtcaggtctttattagcgatgcgggagagatcatcttcaagcgccgaaagctcaagccgacacacgtcgaacgtacgctctatggcgaaggagatgggtctgatttccgcgtcgtcgaaagcagcgtcggacgtctcggagccttgtgctgcgccgagcacattcagccgttgtcgaaatacgccatgtactcgatgaacgagcaagttcacgtggcgtcgtggccatcttttacgctgtatcgcgacaaggcctacgctttgggccatgaggtgaatctcgccgccagccagatctacgcgctagagggaggttgcttcgtcctgcatgcctcggcaattactggtcaagatatgttcgacatgctttgcgatactccggaaaaggccgatttgctgaacgcggagggagcgaagccgggtggaggctattcgatgatcttcggtcccgatggtcagccgatgtgcgagcatctgccgcaggacaaggaaggcatcctctatgctgacgtggacctgtcaatgatcgcaatcgccaaagcggcctacgaccccacggggcattacgcccgcggcgatgtcgtccgtctcatggtcaatcgcagcccgcgtcgcacgagcgtcagcttcagcgaagacgagaacgcggcggtcactttcaccgagacttga

SEQ ID NO: 2 - protein, 334 aa, Bradyrhizobium japonicum
MQDTKFKVAVVQAAPVFMDAPASVAKAIGFIAEAGAAGAKLLAFPEVWIPGYPWWLWLGTPAWGMQFVPRYHANSLRADGPDILALCAAAAEAKINVVMGFSEIDGGTLYLSQVFISDAGEIIFKRRKLKPTHVERTLYGEGDGSDFRVVESSVGRLGALCCAEHIQPLSKYAMYSMNEQVHVASWPSFTLYRDKAYALGHEVNLAASQIYALEGGCFVLHASAITGQDMFDMLCDTPEKADLLNAEGAKPGGGYSMIFGPDGQPMCEHLPQDKEGILYADVDLSMIAIAKAAYDPTGHYARGDVVRLMVNRSPRRTSVSFSEDENAAVTFTET

SEQ ID NO: 3 - DNA, 31 bp, Artificial Sequence
  Description of artificial sequence Bradyrhizobium japonicum USDA110 bll6402 forward PCR primer
tcgcatatgcaggacacgaaattcaaagtcg

SEQ ID NO: 4 - DNA, 28 bp, Artificial Sequence
  Description of artificial sequence Bradyrhizobium japonicum USDA110 bll6402 reverse PCR primer
aaactcgagagtctcggtgaaagtgacc

SEQ ID NO: 5 - DNA, 1002 bp, Artificial Sequence
  Description of artificial sequence bll6402 optimized for expression in E. coli using DNA Star
atgcaggacaccaaattcaaagttgctgttgttcaggctgctccggttttcatggacgctccggcttctgttgctaaagctatcggtttcatcgctgaagctggtgctgctggtgctaaactgctggctttcccggaagtttggatcccgggttacccgtggtggctgtggctgggtaccccggcttggggtatgcagttcgttccgcgttaccacgctaactctctgcgtgctgacggtccggacatcctggctctgtgcgctgctgctgctgaagctaaaatcaacgttgttatgggtttctctgaaatcgacggtggtaccctgtacctgtctcaggttttcatctctgacgctggtgaaatcatcttcaaacgtcgtaaactgaaaccgacccacgttgaacgtaccctgtacggtgaaggtgacggttctgacttccgtgttgttgaatcttctgttggtcgtctgggtgctctgtgctgcgctgaacacatccagccgctgtctaaatacgctatgtactctatgaacgaacaggttcacgttgcttcttggccgtctttcaccctgtaccgtgacaaagcttacgctctgggtcacgaagttaacctggctgcttctcagatctacgctctggaaggtggttgcttcgttctgcacgcttctgctatcaccggtcaggacatgttcgacatgctgtgcgacaccccggaaaaagctgacctgctgaacgctgaaggtgctaaaccgggtggtggttactctatgatcttcggtccggacggtcagccgatgtgcgaacacctgccgcaggacaaagaaggtatcctgtacgctgacgttgacctgtctatgatcgctatcgctaaagctgcttacgacccgaccggtcactacgctcgtggtgacgttgttcgtctgatggttaaccgttctccgcgtcgtacctctgtttctttctctgaagacgaaaacgctgctgttaccttcaccgaaacc

(19) United States
(12) Patent Application Publication
(10) Pub. No.: US 2010/0137636 A1
(43) Pub. Date: Jun. 3, 2010

(54) IDENTIFICATION OF A NITRILASE FROM B. JAPONICUM BY RATIONAL GENOME MINING AND METHODS OF USE

(76) Inventors: Dunming Zhu, Dallas, TX (US); Ling Hua, Dallas, TX (US); Edward R. Biehl, Dallas, TX (US); Chandrani Mukherjee, Chadernagore (IN)

Correspondence Address: BAKER BOTTS L.L.P., 2001 ROSS AVENUE, SUITE 600, DALLAS, TX, 75201-2980, US

(21) Appl. No.: 12/481,092

(22) Filed: Jun. 9, 2009

Related U.S. Application Data
(63) Continuation of application No. 11/608,077, filed on Dec. 7, 2006, now abandoned.
(60) Provisional application No. 60/748,451, filed on Dec. 7, 2005.

(51) Int. Cl. C12P 7/40; C12N 9/78; C12P 13/00; C07C 229/04; C07C 53/00

(52) U.S. Cl. 562/507; 435/136; 435/227; 435/128; 562/553

(57) ABSTRACT

The present disclosure relates to methods of rational genome mining. A method may include narrowing the number of clones that would otherwise need to be screened and/or identifying a gene with a desired catalytic activity. The disclosure also relates to a nitrile hydrolase from Bradyrhizobium japonicum USDA110 first identified by rational genome mining. In addition, the disclosure relates to nitrilase bll6402 and catalytically active variants capable of converting an α-hydroxy nitriles, a β-hydroxy nitrile and/or an α,ω-dinitrile to a carboxylic acid.

### RELATED APPLICATION

This application claims the benefit, under 35 U.S.C. §119(e), of previously filed provisional application entitled Cloning and expression of a nitrilase from B. japonicum, U.S. Application Ser. No. 60/748,451, filed Dec. 7, 2005, the entire contents of which are incorporated herein in their entirety by reference.

### TECHNICAL FIELD

The present disclosure relates to a nitrilase with desirable catalytic activity. The present invention also relates to a rational genome mining approach to identifying an efficient enzyme catalyst for a target transformation.

### BACKGROUND OF THE INVENTION

With ever-increasing environmental concerns, development of “green” methods to produce fine chemicals are highly desirable. Biocatalysis accommodates several of the twelve principles of green chemistry defined by Anastas and Warner (1998. Green Chemistry: Theory and Practice. Oxford University Press, New York). Intensive efforts have been made to discover new enzyme catalysts in both academia and industry. Various approaches may be developed and used to discover or evolve novel enzymes. Examples of these include directed evolution and metagenomic approaches, but these may require screening a large number of clones. Accordingly, enzyme catalyst identification for target transformation may be time consuming and, consequently, may present a critical bottleneck in the enzyme catalyst discovery.

### SUMMARY OF THE INVENTION

Thus, a need has arisen for efficient methods for identifying an enzyme catalyst. Accordingly, in some embodiments, the present disclosure relates to a rational genome mining approach to discovery of an efficient enzyme catalyst for target transformation. A method of rational genome mining may include a genomic approach to identifying a nucleic acid comprising a sequence encoding an enzyme having a desired substrate and/or product specificity. In some embodiments, for example, a method of identifying a nucleic acid comprising a sequence encoding an enzyme having a desired substrate and/or product specificity may include (a) selecting a genome mining database query selected from the group consisting of a text-based query, a sequence-based query, and combinations thereof, (b) running the genome mining database query against a database comprising a nucleotide sequence or series of nucleotide sequences that, in the aggregate, correspond to at least 75% of the genome of an organism, to produce at least one candidate nucleotide sequence, (c) examining at least one nucleotide sequence flanking each at least one candidate nucleotide sequence and determining whether the at least one flanking nucleotide sequence comprises an open reading frame or a predicted open reading frame, and (d) determining whether the polypeptide encoded by each actual or predicted open reading frame is operable to produce or predicted to be operable to the desired substrate or a derivative thereof and/or consume the desired product or a derivative thereof, wherein a candidate nucleotide sequence is identified as encoding an enzyme having the desired substrate and/or product specificity where at least one flanking nucleotide sequence comprises an actual or predicted open reading frame encoding a polypeptide that is operable to produce or predicted to be operable to the desired substrate or a derivative thereof and/or consume the desired product or a derivative thereof.

The disclosure relates, in some embodiments, to methods for predicting whether a nucleic acid comprises a sequence encoding an enzyme having a desired substrate and/or product specificity. For example, a method may include (a) selecting a genome mining database query selected from the group consisting of a text-based query, a sequence-based query, and combinations thereof, (b) running the genome mining database query against a database comprising a nucleotide sequence or series of nucleotide sequences that, in the aggregate, correspond to at least 75% of the genome of an organism, to produce at least one candidate nucleotide sequence, (c) examining at least one nucleotide sequence flanking each at least one candidate nucleotide sequence and determining whether the at least one flanking nucleotide sequence comprises an open reading frame or a predicted open reading frame, and (d) determining whether the polypeptide encoded by each actual or predicted open reading frame is operable to produce or predicted to be operable to the desired substrate or a derivative thereof and/or consume the desired product or a derivative thereof, wherein a candidate nucleotide sequence is predicted to encode an enzyme having the desired substrate and/or product specificity where at least one flanking nucleotide sequence comprises an actual or predicted open reading frame encoding a polypeptide that is operable or predicted to be operable to produce the desired substrate or a derivative thereof and/or consume the desired product or a derivative thereof.

A rational genome mining method, according to some embodiments of the disclosure, may reduce the number of clones to be screened by more than about 10%, more than about 25%, more than about 50%, more than about 75%, more than about 90%, or more than about 95% compared to a method of genome mining that does not consider the presence and function of nearby genes.

The disclosure also relates to a nitrilase (e.g., from Bradyrhizobium japonicum) and catalytically active variants thereof. In some embodiments, a nitrilase (e.g., nitrilase bll6402) may have catalytic activity over a broad range of temperature (e.g., up to about 50° C.) and/or pH (e.g., from about 5 to about 9). A nitrilase (e.g., nitrilase bll6402) may also have, in some embodiments, catalytic activity in the presence of one or more organic solvents. According to some embodiments, a nitrilase (e.g., nitrilase bll6402) may convert α-hydroxynitriles to α-carboxylic acids with a Vₘₐₓ from about 0.001 U/mg to about 100 U/mg and/or a Kₘ from about 10 μM to about 10 mM. For example, a Vₘₐₓ for this conversion may be from about 0.001 U/mg to about 0.010 U/mg, from about 0.010 U/mg to about 0.100 U/mg, from about 0.100 U/mg to about 1.0 U/mg, from about 1.0 U/mg to about 10.0 U/mg, or from about 10.0 U/mg to about 100.0 U/mg. Similarly, a Kₘ for this conversion may be from about 10 μM to about 100 μM, from about 100 μM to about 1 mM, or from about 1.0 mM to about 10 mM.

According to some embodiments, a nitrilase (e.g., nitrilase bll6402) may convert β-hydroxynitriles to β-carboxylic acids with a Vₘₐₓ from about 0.001 U/mg to about 100 U/mg and/or a Kₘ from about 10 μM to about 10 mM. For example, a Vₘₐₓ for this conversion may be from about 0.001 U/mg to about 0.010 U/mg, from about 0.010 U/mg to about 0.100 U/mg, from about 0.100 U/mg to about 1.0 U/mg, from about 1.0 U/mg to about 10.0 U/mg, or from about 10.0 U/mg to about 100.0 U/mg. Similarly, a Kₘ for this conversion may be from about 10 μM to about 100 μM, from about 100 μM to about 1 mM, or from about 1.0 mM to about 10 mM.

According to some embodiments, a nitrilase (e.g., nitrilase bll6402) may convert α,ω-dinitriles to ω-carboxylic acids with a Vₘₐₓ from about 0.001 U/mg to about 100 U/mg and/or a Kₘ from about 10 μM to about 10 mM. For example, a Vₘₐₓ for this conversion may be from about 0.001 U/mg to about 0.010 U/mg, from about 0.010 U/mg to about 0.100 U/mg, from about 0.100 U/mg to about 1.0 U/mg, from about 1.0 U/mg to about 10.0 U/mg, or from about 10.0 U/mg to about 100.0 U/mg. Similarly, a Kₘ for this conversion may be from about 10 μM to about 100 μM, from about 100 μM to about 1 mM, or from about 1.0 mM to about 10 mM.

A nitrilase according to some embodiments of the disclosure (e.g., nitrilase bll6402) may catalyze the hydrolysis of various aliphatic nitriles with diverse structures and/or the selective hydrolysis of α,ω-dinitriles. Selectivity in the hydrolysis of α,ω-dinitriles may be independent of chain length.

In some embodiments, a nitrilase of the disclosure may be isolated and/or purified. A nitrilase may include a polypeptide having the amino acid sequence of SEQ ID NO: 2. A nitrilase, according to some embodiments, may comprise a polypeptide having an amino acid sequence that is more than about 95% identical to SEQ ID NO: 2 and/or having a catalytic activity sufficient to convert at least about 50% of a nitrile to a carboxylic acid at a pH between about 5 and about 9 and a temperature of less than about 50° C.

In some embodiments, a nitrilase may have catalytic activity in a biphasic solvent. For example, a nitrilase may have catalytic activity in a solvent comprising water and an organic solvent (e.g., hexane, tert-butyl methyl ether, and/or toluene). A nitrilase may have more than about 50%, more than about 60%, or more than about 70% of the catalytic activity in a biphasic solvent compared to its catalytic activity in an aqueous solvent. A biphasic solvent may include up to about 10% (V/V) organic solvent, up to about 20% (V/V) organic solvent, up to about 30% (V/V) organic solvent, or up to about 40% (V/V) organic solvent.

A purified nitrilase, according to some embodiments, may comprise a polypeptide having an amino acid sequence that is more than about 95% identical to SEQ ID NO: 2, and/or have a catalytic activity sufficient to convert, in a solvent at a pH between about 5 and about 9 and a temperature of less than about 50° C., at least about 50% of a nitrile to a carboxylic acid. A solvent may be, for example, an aqueous solvent or a biphasic solvent. A biphasic solvent may include water and an organic solvent (e.g., up to about 30% (V/V)). An organic solvent may be selected from the group consisting of dimethyl sulfoxide, tert-butyl methyl ether, hexane, toluene, butyl acetate, and combinations thereof.

A nitrile susceptible to a nitrilase of the disclosure may include, for example, an α-hydroxy nitrile (e.g., aromatic α-hydroxy nitrile), a β-hydroxy nitrile, and/or an α,ω-dinitrile. A nitrile may include mandelonitrile, α-trimethylsilyloxyphenylacetonitrile, 2-phenylglycinonitrile, α-n,n-dimethylaminophenylacetonitrile, α-phenylpropionitrile, α-phenylbutyronitrile, phenylacetonitrile, hydrocinnamonitrile, 4-phenylbutyronitrile, 3-indolylacetonitrile, benzonitrile, 4-acetylbenzonitrile, n-butyronitrile, 4-chlorobutyronitrile, valeronitrile, hexanenitrile, heptanenitrile, crotononitrile, allyl cyanide, 1,4-dicyanobutane, 2-methylglutaronitrile, 3-aminopropionitrile, 3-hydroxypropionitrile, and/or methylthioacetonitrile. A nitrile may include 2-trimethylsilyloxy-2-phenylacetonitrile, 2-phenylpropionitrile, 2-phenylbutyronitrile, phenylacetonitrile, hydrocinnamonitrile, 4-phenylbutyronitrile, benzonitrile, n-butyronitrile, valeronitrile, hexanenitrile, heptanenitrile, crotonitrile, allyl cyanide, and/or methylthioacetonitrile.

In some embodiments, a carboxylic acid that may be produced by a nitrilase of the disclosure may include α-hydroxycarboxylic acid, β-hydroxycarboxylic acid, and/or ω-cyanocarboxylic acid. For example, a carboxylic acid may include mandelic acid, α-hydroxy-α-(p-hydroxyphenyl)acetic acid, α-hydroxy-α-(m-hydroxyphenyl)acetic acid, 2-phenylpropionic acid, phenylacetic acid, hydrocinnamic acid, 4-phenylbutyric acid, benzoic acid, butyric acid, valeronic acid, hexanoic acid, heptanoic acid, crotonic acid, 3-butenoic acid, and/or methylthioacetic acid.

In some embodiments, a nucleic acid encoding a nitrilase of the disclosure may be isolated and/or purified. A nucleic acid encoding a nitrilase according to some embodiments may include a nucleic acid sequence that is more than about 60%, more than about 75%, more than about 90%, more than about 97% identity to SEQ ID NO: 1. A nitrilase encoded by such a nucleic acid may have a catalytic activity sufficient to convert at least about 50% of a nitrile to a carboxylic acid at a pH between about 5 and about 9 and a temperature of less than about 50° C.

A nitrile susceptible to a nitrilase encoded by a nucleic acid having more than about 60% identity to SEQ ID NO: 1 may include, for example, an α-hydroxy nitrile (e.g., aromatic α-hydroxy nitrile), a β-hydroxy nitrile, and/or an α,ω-dinitrile. A nitrile may include mandelonitrile, α-trimethylsilyloxyphenylacetonitrile, 2-phenylglycinonitrile, α-n,n-dimethylaminophenylacetonitrile, α-phenylpropionitrile, α-phenylbutyronitrile, phenylacetonitrile, hydrocinnamonitrile, 4-phenylbutyronitrile, 3-indolylacetonitrile, benzonitrile, 4-acetylbenzonitrile, n-butyronitrile, 4-chlorobutyronitrile, valeronitrile, hexanenitrile, heptanenitrile, crotononitrile, allyl cyanide, 1,4-dicyanobutane, 2-methylglutaronitrile, 3-aminopropionitrile, 3-hydroxypropionitrile, and/or methylthioacetonitrile. A nitrile may include 2-trimethylsilyloxy-2-phenylacetonitrile, 2-phenylpropionitrile, 2-phenylbutyronitrile, phenylacetonitrile, hydrocinnamonitrile, 4-phenylbutyronitrile, benzonitrile, n-butyronitrile, valeronitrile, hexanenitrile, heptanenitrile, crotonitrile, allyl cyanide, and/or methylthioacetonitrile.

A carboxylic acid that may be produced by a nitrilase encoded by a nucleic acid having more than about 60% identity to SEQ ID NO: 1 may include, for example, α-hydroxycarboxylic acid, β-hydroxycarboxylic acid, and/or ω-cyanocarboxylic acid. For example, a carboxylic acid may include mandelic acid, α-hydroxy-α-(p-hydroxyphenyl)acetic acid, α-hydroxy-α-(m-hydroxyphenyl)acetic acid, 2-phenylpropionic acid, phenylacetic acid, hydrocinnamic acid, 4-phenylbutyric acid, benzoic acid, butyric acid, valeronic acid, hexanoic acid, heptanoic acid, crotonic acid, 3-butenoic acid, and/or methylthioacetic acid.

The disclosure also relates to methods of converting a nitrile to a carboxylic acid. In some embodiments, a method of converting a nitrile to a carboxylic acid may include contacting a nitrile with a nitrilase under conditions that permit catalysis (e.g., hydrolysis) wherein the nitrilase (a) comprises a polypeptide having an amino acid sequence that is more than about 95%, more than about 97%, or more than about 99% identical to SEQ ID NO: 2 and/or (b) has a catalytic activity sufficient to convert at least 50% of a nitrile to a carboxylic acid at a pH between about 5 and about 9 and a temperature of less than about 50° C.

In addition, the disclosure relates to methods of converting a β-hydroxy nitrile to a corresponding β-hydroxycarboxylic acid. In some embodiments, a method of converting a β-hydroxy nitrile to a β-hydroxycarboxylic acid may include contacting a β-hydroxy nitrile with a nitrilase under conditions that permit catalysis (e.g., hydrolysis) wherein the nitrilase (a) comprises a polypeptide having an amino acid sequence that is more than about 95%, more than about 97%, or more than about 99% identical to SEQ ID NO: 2 and/or (b) has a catalytic activity sufficient to convert at least 50% of a nitrile to a carboxylic acid at a pH between about 5 and about 9 and a temperature of less than about 50° C.

The disclosure further relates to methods of converting a α,ω-dinitrile to a corresponding ω-cyanocarboxylic acid. In some embodiments, a method of converting a α,ω-dinitrile to a ω-cyanocarboxylic acid may include contacting a α,ω-dinitrile with a nitrilase under conditions that permit catalysis (e.g., hydrolysis) wherein the nitrilase (a) comprises a polypeptide having an amino acid sequence that is more than about 95%, more than about 97%, or more than about 99% identical to SEQ ID NO: 2 and/or (b) has a catalytic activity sufficient to convert at least 50% of a nitrile to a carboxylic acid at a pH between about 5 and about 9 and a temperature of less than about 50° C. In some embodiments, a nitrilase have a catalytic activity for converting a α,ω-dinitrile to a corresponding ω-cyanocarboxylic acid that is independent of chain length.

The disclosure relates to methods of producing a nitrilase operable to convert at least about 50% of a nitrile to a carboxylic acid at a pH between about 5 and about 9 and a temperature of less than about 50° C. According to some embodiments of the disclosure, a method of producing a nitrilase may include contacting a organism with a nucleic acid comprising an expression control sequence operably linked to a nucleotide sequence encoding a polypeptide having the amino acid sequence of SEQ ID NO: 2 under conditions that permit expression of the nucleotide sequence and production of the nitrilase. An organism suitable for producing a nitrilase may be a plant or microorganism. Non-limiting examples of suitable microorganisms may include Escherichia, Bradyrhizobium, Bacillus, baculovirus, and yeast.

### BRIEF DESCRIPTION OF THE DRAWINGS

Some specific example embodiments of the disclosure may be understood by referring, in part, to the description and the accompanying drawings, wherein:

FIG. 1 is a line drawing showing the organization of the Bradyrhizobium japonicum genome near bll6402;

FIG. 2 shows a sequence similarity tree based on a sequence alignment performed by Clustal V of Lasergene program from DNAStar (the nitrilases from different organisms are annotated as following: Acidovorax facilis 72W (Afac72W), Rhodococcus rhodochrous J1 (RrhJ1), Rhodococcus rhodochrous K22 (RrhK22), Klebsiella ozaenae (Koza), Comamonas testosterone (Ctes), Pseudomonas fluorescens EBC191 (PfluEBC191), Alcaligenes faecalis JM3 (AfaeJM3), Synechocystis sp. PCC 6803 (PCC6803), Bradyrhizobium japonicum USDA (bll6402));

FIG. 3 illustrates temperature dependence of hydrolytic activity of an example nitrilase of the disclosure on phenylacetonitrile measured at pH=7.1 with different reaction time intervals of 1 hr (diamond); 2 hrs (square); 4 hrs (triangle); 16 hrs (cross);

FIG. 4 illustrates pH dependence of hydrolytic activity of an example nitrilase of the disclosure on phenylacetonitrile measured at 30° C. with different reaction time intervals of 1 hr (diamond); 2 hrs (square); 4 hrs (triangle); 16 hrs (cross); and

FIG. 5 illustrates organic solvent effects on hydrolytic activity of an example nitrilase of the disclosure on phenylacetonitrile measured at 30° C. and pH=7.10 with different organic solvents (1. No organic solvent; 2.10% DMSO; 3.20% DMSO; 4.30% hexane; 5.30% toluene; 6.30% MTBE; 7.30% butyl acetate).

### DETAILED DESCRIPTION OF THE INVENTION

Advances in molecular biology have enabled the complete sequencing of hundreds of microorganism genomes. The rapidly growing microbial genome sequence data obtained in the course of genome projects offer a tremendous opportunity for discovery of new enzyme catalysts. According to some embodiments of the present disclosure, this information may be harnessed through rational genome mining. Rational genome mining may include (a) genome mining (e.g., traditional genome mining) that yields one or more candidate putative gene and (b) functional analysis of the genetic organization of one or more candidate putative genes within a microorganism's chromosome. For example, an enzyme of desired substrate and/or product specificity may be identified by creating a nucleic acid sequence database query and applying the query to a database comprising at least one microorganism genome and/or at least one substantially complete microorganism genome to produce one or more candidate nucleic acid sequences. A query may include the name of the desired enzyme (e.g., “nitrilase”) and/or a nucleic acid or amino acid sequence (e.g., a consensus sequence or example sequence of known activity).

Nucleic acid sequences flanking each candidate nucleic acid sequence may be analyzed to determine whether ORFs are present and, if so, what polypeptides and/or proteins may be encoded by these ORFs. A candidate nucleic acid sequence may be identified as encoding a nitrilase of desired substrate and/or product specificity where one or more of its flanking nucleic acid sequences encode an enzyme that may produce the desired substrate and or operate on the desired product (e.g., based on empirical data or sequence-based prediction).

Aromatic α-hydroxy carboxylic acids may be valuable intermediates in pharmaceutical and fine chemical industry. For example, p-hydroxymandelic acid may be used as a chemical adaptor system to link a tumor-targeting device with a prodrug and an enzymatic trigger for selective chemotherapy. A straightforward method to produce α-hydroxy carboxylic acids may include hydrolysis of a corresponding α-hydroxy nitriles. An α-hydroxy nitrile may be prepared, in turn, by addition of HCN to a corresponding aldehyde (Scheme 1).

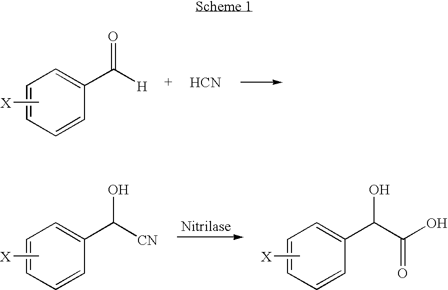

Chemical hydrolysis of nitriles to carboxylic acids may require drastic conditions (e.g., strong bases or acids and relatively high reaction temperature), and may produce unwanted byproducts and/or large amounts of inorganic wastes. According to some embodiments of the disclosure, however, the need for drastic conditions is reduced or eliminated. For example, a nitrile may be biotransformed to a carboxylic acid at a pH from about 5 to about 9 and/or a temperature below about 60° C. A nitrile may be biotransformed to a carboxylic acid at a pH from about 6 to about 8 and/or a temperature from about 10° C. to about 50° C. A nitrile may be biotransformed to a carboxylic acid at a pH from about 6.5 to about 7.5 and/or a temperature from about 30° C. to about 50° C. In some example embodiments, a nitrilase may hydrolyze an aromatic α-hydroxy nitrile to an α-hydroxy carboxylic acid at about pH 7.2 and at about 30° C. In some example embodiments, a nitrilase may hydrolyze a β-hydroxy nitrile to a β-hydroxy carboxylic acid at about pH 7.2 and at about 30° C. In some example embodiments, a nitrilase may hydrolyze α,ω-dinitriles to w-cyanocarboxylic acids at about pH 7.2 and at about 30° C.

In view of the fact that bacterial genes may cluster based on linked functions, the genetic organization of a putative nitrilase gene within a chromosome(s) of a microbe may shed some light on the natural function of the encoded enzymes. Accordingly, the function or putative function of one or more open reading frames near each candidate nitrilase-encoding nucleic acid sequence may be considered. For example, flanking (e.g., adjacent) open reading frames may be considered. Open reading frames may be deemed to be near enough for consideration if they are within about 30,000 bases, within about 20,000 bases, within about 10,000 bases, within about 5,000 bases, within about 2,500 bases, within about 1,000 bases, and/or within about 300 bases. Open reading frames may be on the same strand or a complimentary strand. In some embodiments, it may even be deemed helpful or desirable to consider regions on homologous (or other) chromosomes.

In some embodiments of the disclosure, an enzyme with a desired specificity may be identified in a microbe. Many microbes, particularly those with an ecological relationship with plants and animals, metabolize cyanosugars, cyanohormones, and other nitrile compounds. Cyanogenic glycosides derived from amino acids are the most abundant group of nitrile compounds present in plants. Cyanogenic glycosides may be metabolized through cyanohydrin intermediates that may be decomposed to aldehydes and HCN by oxynitrilases in plants.

Bradyrhizobium japonicum USDA110, originally isolated from soybean nodule in Florida, is a gram-negative nitrogen-fixing microbe. This symbiont provides its host plant with fixed nitrogen and, in return, receives a steady supply of carbon and shelter. O-glycosylated mandelonitrile (A) may also be secreted to Bradyrhizobium japonicum by symbiotic interaction and metabolized through an alternative pathway (Scheme 2). In this metabolic pathway, O-glycosylated mandelonitrile (A) may be cleaved by β-glucosidase (bll6177) to mandelonitrile, which may be detoxified by nitrilase (bll6402) to yield mandelic acid and ammonia. The resulting mandelic acid may be then oxidized by mandelate dehydrogenase and follow the pathway as shown in Scheme 2. A similar metabolic pathway for cyanogenic glycosides, in which cyanogenic glycosides are cleaved with the action of β-glucosidase to α-hydroxy nitrile compounds followed by hydrolysis catalyzed by nitrile hydratase and amidase, has been proposed in Brevibacterium.

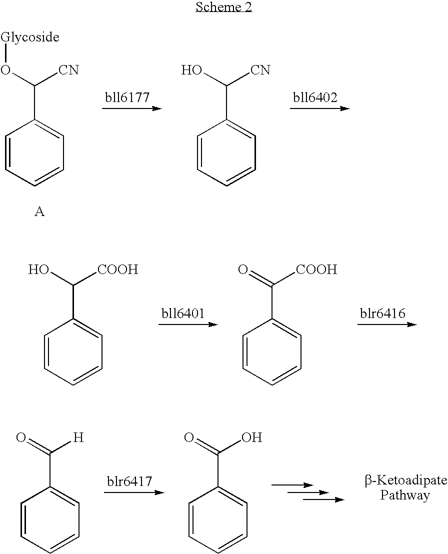

In some embodiments, a nitrilase (or other enzyme of interest) may be identified by in silico screening one or more microorganism DNA sequence databases. Genome sequences may be compared with those of known nitrilase genes (query sequences) to identify one or more putative nitrilase-encoding sequences based on sequence homology and/or identity. Many open reading frames (ORFs) encoding putative nitrilases may be identified in sequenced microbial genomes. However, it may not be clear initially which putative nitrilase possesses a desired activity, e.g., hydrolyzing aromatic α-hydroxy nitriles to carboxylic acids. Using this approach allowed bll6402 to be identified as a putative nitrilase gene (Examples 1-7). In some embodiments, to ascertain what activity, if any, a putative nitrilase gene (e.g., bll6402) from Bradyrhizobium japonicum USDA110 may have, nearby sequences may be evaluated.

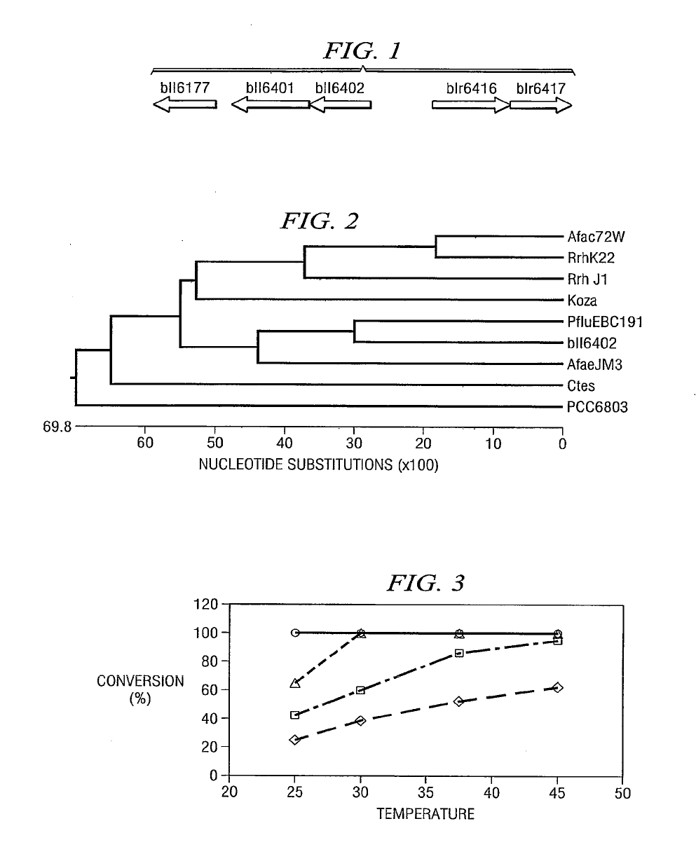

As shown in FIG. 1, the ORFs surrounding bll6402 include several putative genes in an aromatic catabolic pathway. These genes are putative (S)-mandelate dehydrogenase (bll6401), benzoylformate decarboxylase (blr6416), benzaldehyde dehydrogenase (blr6417), and α-glucosidase (bll6177).

Without being limited to any particular mechanism of action, this suggests that the predicted mandelonitrile metabolic pathway in Scheme 2 might exist in B. japonicum and the natural function of nitrilase bll6402 might be to detoxify and utilize hydroxynitriles produced in the metabolism of cyanogenic glycosides. In any case, the presence of these flanking ORFs identified bll6402 as potentially encoding a nitrilase having mandelonitrile as a substrate (e.g., a native substrate) and/or capable of hydrolyzing aromatic α-hydroxy nitriles to carboxylic acids.

To further characterize bll6402, this putative nitrilase gene was cloned and expressed in E. coli, and the encoded protein was purified to give a nitrilase with a molecular mass of about 37 kD. The molecular weight of the holoenzyme was about 455 kD, suggesting that nitrilase bll6402 may self-aggregate to the active form with a native structure being 12 subunits of identical size. This nitrilase was most active toward mandelonitrile with Vₘₐₓ and Kₘ for mandelonitrile being 44.7 U/mg and 0.26 mM, respectively. The k_{cat} and overall catalytic efficiency k_{cat}/Kₘ were 27.0 s⁻¹ and 1.04×10⁵ M⁻¹s⁻¹, indicating that nitrilase bll6402 may be very active for the hydrolysis of mandelonitrile to mandelic acid. Nitrilase bll6402 also effectively hydrolyzed several mandelonitrile derivatives.

In some embodiments, nitrilase bll6402 may catalyze the enantioselective hydrolysis of β-hydroxy nitriles to (S)-enriched β-hydroxy carboxylic acid and/or selectively hydrolyze α,ω-dinitriles to α-cyanocarboxylic acids. For example, nitrilase bll6402 may hydrolyze 1-cyanocyclohexaneacetonitile to 1-cyanocyclohexaneacetic acid, which may be a precursor for a drug with anti-depressant properties (e.g., gabapentin (a.k.a., Neurontin)) (Scheme 3).

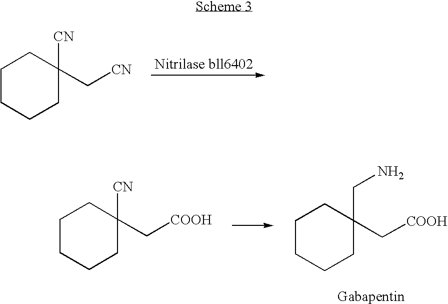

In some embodiments, nitrilase bll6402 may catalyze a reaction related to synthesis of a drug with anti-convulsant properties (e.g., pregabalin (a.k.a. Lyrica). (Scheme 4)

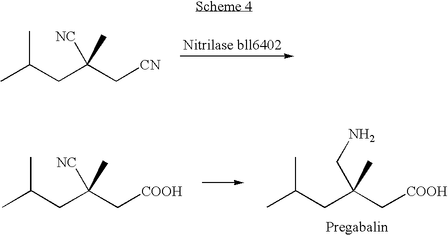

In some embodiments, nitrilase bll6402 may catalyze a reaction related to synthesis of a cholesterol lowering drug (e.g., atorvastatin (a.k.a. Lipitor)) (Scheme 5).

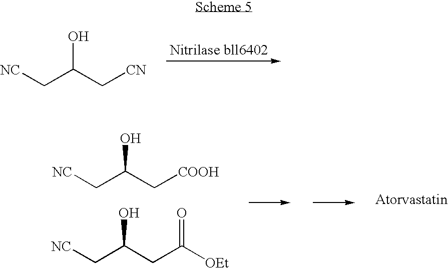

A nitrilase, according to some embodiments of the disclosure, may include a polypeptide having the amino acid sequence of SEQ ID NO: 2 and/or variants thereof. For example, a nitrilase capable of hydrolyzing nitriles to carboxylic acids with a Vₘₐₓ of at least about 0.001 U/mg and/or a Kₘ below about 10 mM may include a polypeptide with an amino acid sequence that is more than about 60%, more than about 70%, more than about 80%, more than about 85%, more than about 90%, more than about 95%, more than about 97%, more than about 98%, or more than about 99% identical to SEQ ID NO: 2. In some embodiments, a nitrilase may include a polypeptide having an amino acid sequence that is more than about 95% identical to SEQ ID NO: 2 and/or may have a catalytic activity sufficient to convert at least about 50% of a nitrile to a carboxylic acid at a pH from about 5 to about 9 and a temperature less than 60° C.

A nitrilase, according to some embodiments of the disclosure, may be encoded by a nucleic acid (e.g., DNA or RNA) comprising a nucleic acid having the sequence of SEQ ID NO: 1 and/or variants thereof. For example, a nitrilase capable of hydrolyzing nitriles to carboxylic acids with a Vₘₐₓ of at least about 0.001 U/mg and/or a Kₘ below about 10 mM may be encoded by a nucleic acid comprising a nucleic acid having a sequence that is more than about 60%, more than about 70%, more than about 75%, more than about 78%, more than about 80%, more than about 85%, more than about 90%, more than about 95%, more than about 97%, more than about 98%, or more than about 99% identical to SEQ ID NO: 1. In some embodiments, a nitrilase may be encoded by a nucleic acid comprising a nucleotide sequence that is more than about 90% identical to SEQ ID NO: 1 and/or may have a catalytic activity sufficient to convert at least about 50% of a nitrile to a carboxylic acid at a pH from about 5 to about 9 and a temperature less than 60° C.

In some embodiments, a nucleic acid encoding a nitrilase may be designed by modifying SEQ ID NO: 1 to optimize codon usage in an organism (e.g., a desired host organism). For example, Bradyrhizobium codons that are rare, inefficient, and/or error-prone in a desired host organism (e.g., E. coli) may be replaced with codons more suited to the host. A codon optimized nitrilase bll6402 sequence may include, for example, SEQ ID NO: 5, which is about 78% identical to SEQ ID NO: 1.

A nucleic acid encoding a nitrilase, in some embodiments, may be designed by modifying SEQ ID NO: 1 to reduce or eliminate sequences that may adversely affect a desired expression level in a host organism. For example, if SEQ ID NO: 1 includes one or more sequences that may operate as expression control sequences in a particular host, these sequences may be modified or removed, at least to the extent that the modified sequence still encodes a nitrilase with a desired catalytic activity.

### EXAMPLES

Some embodiments of the disclosure may be illustrated by one or more of the following examples.

### Example 1

### Identification of a Putative Nitrilase Gene bll6402) by Rational Genome Mining

An in silico screening of DNA sequence a database for putative nitrilase genes in microorganisms was performed. A query using “nitrilase” as an identifier was submitted to Entrez Gene database and 98 hits were obtained (as of March 2005). Since bacteria were known to be responsible for the degradation of many nitrile compounds, non-bacterial hits were excluded reducing the number of hits to 51. Among them, 49 ORFs (open reading frames) were annotated as possibly encoding proteins containing a carbon-nitrogen hydrolase domain, which might belong to nitrilase superfamily. The two that did not contain a carbon-nitrogen hydrolase domain were excluded. Since almost all of the known nitrilases consist of from 300 to 385 amino acids, open reading frames predicted to encode proteins outside of this range were excluded. Sixteen ORFs encoding putative nitrilases were identified in 14 sequenced microbial genomes, as presented in Table 1.

Next, the surrounding open reading frames of these 16 putative nitrilase genes were examined and 12 of them were found to be flanked by either hypothetical proteins (i.e., sequences that, if transcribed and translated, yield proteins with no known or predicted function) or regulator/transporter proteins. The other 4 putative nitrilase genes were flanked by putative functional genes. One of the putative nitrilase genes (bll6402) from Bradyrhizobium japonicum USDA110 was found to be next to a putative mandelate dehydrogenase (bll6401) which was 55.2% identical to (S)-mandelate dehydrogenase from P. putia. BLL6402 was also surrounded by two other putative genes, the first encoding benzoylformate decarboxylase (blr6416) and the second encoding benzaldehyde dehydrogenase (blr6417). Both benzoylformate decarboxylase and benzaldehyde dehydrogenase may be in the aromatic catabolic pathway. In addition, BLL6402 was surrounded by a putative gene encoding β-glucosidase (bll6177). This suggested that a mandelonitrile metabolic pathway as shown in Scheme 2 might exist in B. japonicum and mandelonitrile could be the native substrate of the putative nitrilase encoded by bll6402 gene, which might be an effective catalyst for the hydrolysis of aromatic α-hydroxy nitriles to carboxylic acids.

### Example 2

### Cloning and Expression of the Nitrilase Gene bll6402

The Bradyrhizobium japonicum USDA110 strain was obtained from a stock collection maintained by the United States Department of Agriculture's Soybean Genomics and Improvement Laboratory.

The bll6402 gene (nucleotide, SEQ ID NO: 1; amino acid, SEQ ID NO: 2) was amplified from Bradyrhizobium japonicum USDA110 genomic DNA by forward primer 5′-TCGCATATGCAGGACACGAAATTCAAAGTCG-3′ (the Nde I restriction site is underlined) (SEQ ID NO: 3) and reverse primer 5′-AAACTCGAGAGTCTCGGTGAAAGTGACC5-3′ (the Xho I restriction site is underlined) (SEQ ID NO: 4). The amplification was performed in a final volume of 100 μl, and the reaction mixtures contained 200 ng of genomic DNA, 50 μmol of each primer, 200 μM of dNTP, 1×PCR buffer, 1.25 U of Pfx DNA polymerase and 1 mM MgSO₄. The PCR amplified DNA fragment was digested with NdeI and Xho I and the 1026 by insert was cloned into pET22b expression vector digested with the same restriction enzymes. This insert includes SEQ ID NO:1, a his tag, and restriction sites. The resulting plasmid (designated as pLH10.3) was transformed into Rosetta (DE3)pLysS E. coli strain and propagated on LB agar plate containing 100 μg/ml of ampicillin and 34 μg/ml of chloramphenicol. An overnight culture from a single colony was diluted into a fresh LB medium containing 100 μg/ml of ampicillin and 34 μg/ml of chloramphenicol until OD₅₉₅ reached 0.6 to 1.0, and the cells were induced with 0.1 mM of IPTG for 6 hours at 30° C. on an orbital shaker at 180 rpm. The cells were harvested.

### Example 3

### Preparation of Cell Extract and Purification of the Enzyme

The cultures of E. coli Rosetta (DE3)pLysS were harvested by centrifugation. The cell pellet was resuspended in potassium phosphate lysis buffer (10 mM, pH 7.2, 1 mM DTT), and the cells were lysed by homogenizer. The cell-free extract was mixed with equal volume of 2×PEI solution (0.25% polyethyleneimine MW 40K-60K, 6% NaCl, 100 mM Borax, pH 7.4) to remove lipids. The PEI-treated supernatant was precipitated with 45% ammonium sulfate. The resulting precipitate was collected after centrifugation and dissolved in potassium phosphate buffer (10 mM, pH 7.2, 1 mM DTT). The lysate was dialysed by gel filtration into potassium phosphate buffer (10 mM, pH 7.2, 1 mM DTT), and then used for activity assay.

### Example 4

### Purification of the His-Tagged Enzyme

Cell-free extract of E. coli Rosetta (DE3)pLysS expressed bll6402 His-tagged protein was prepared by following the same procedure as described above. The cell-free extract was precipitated with 45% ammonium sulfate, and the resulting precipitate was collected after centrifugation and resuspended in sodium phosphate buffer (50 mM, pH 8.0) containing 15 mM of imidazole and 300 mM of NaCl. The lysate was desalted on econo-pac 10DG column using the same buffer as eluent. The His-tagged nitrilase was then purified in a single step by imidazole metal affinity chromatography. The Ni-NTA slurry (2 ml, Qiagen) was loaded on a column and equilibrated with sodium phosphate buffer (50 mM, pH 8.0) containing 15 mM of imidazole and 300 mM of NaCl. The lysate (7 ml) was added to the column and washed twice with 4 ml of wash buffer (pH 8.0) containing 50 mM of sodium phosphate, 40 mM of imidazole and 300 mM of NaCl. Pure protein was eluted with 3 ml of elution buffer (pH 8.0) containing 50 mM of sodium phosphate, 250 mM of imidazole and 300 mM of NaCl. The fractions containing pure protein were combined and desalted on an econo-pac column with 10 mM potassium phosphate buffer containing 1 mM DTT (pH 7.2), and then stored in 50% glycerol at −20° C.

### Example 5

### Determination of Molecular Mass

To determine the molecular mass of the holoenzyme, size exclusion high-performance liquid chromatography was performed using an Agilent 1100 series high-performance liquid chromatography system with a Superdex 200 10/300 GL column (Amersham Biosciences). The column was calibrated using thyroglobulin (Mᵣ 669,000), ferritin (Mᵣ 440,000), and catalase (Mᵣ 232,000) as references (high-molecular-weight kit from Amersham Biosciences). The eluent was potassium phosphate buffer (50 mM, pH 7.0) with 0.15 M NaCl, and flow rate was 0.4 ml/min at room temperature. The mass of the native protein was calculated from its retention time.

The purified protein gave a single band on SDS-PAGE with a molecular mass of about 37 kD. The molecular weight of the holoenzyme was determined by size exclusion high-performance liquid chromatography to be about 455 kD. This suggested that nitrilase bll6402 self-aggregated to the active form with native structure being 12 subunits of identical size. Most known nitrilases consist of a single polypeptide with a molecular mass in the range of 32-45 kD, which self-associate to form active enzymes. The preferred native form of nitrilases seems to be a large aggregate of 6-26 units. The present observation is consistent with the known nitrilases.

### Example 6

### Measurement of Kinetic Parameters

The standard assays were carried out by mixing the substrate nitrile and purified nitrilase enzyme in potassium phosphate buffer (100 mM, pH 7.2). The reaction mixture was incubated at 30° C. Samples (100 μL) of the reaction mixture were withdrawn at several time intervals and quenched with 10 μl of 1 M HCl. The amount of ammonia produced in the reaction was measured using Bertholet assay as follows: A 100 μL aliquot of each of the following assay reagents was added subsequently: 0.02 M NaOCl, 0.33 M sodium phenoxide and 0.01% sodium nitroprusside solution. The mixture was then heated at 95° C. for 2 min, cooled rapidly in ice-water bath, and 200 μl of the mixture was then placed in a 96-well plate to measure the absorbance at 640 nm using SpectraMax M2 microplate reader (Molecular Devices). The amount of ammonia released during the hydrolysis was calculated by comparison with the standard curve obtained with standard ammonium chloride solution, and the activity was defined as the number of μmol of ammonia produced in 1 minute by 1 mg of enzyme (μmol·min⁻¹·mg⁻¹). To determine the kinetic parameters of the purified protein for mandelonitrile, activity assays were performed with different substrate concentrations, and Vₘₐₓ and Kₘ were calculated by best fitting the curve to experimental data according to Michaelis equation.

The obtained Vₘₐₓ and Kₘ. for mandelonitrile were 44.7 U/mg and 0.26 mM, respectively. The k_{cat} and overall catalytic efficiency k_{cat}/Kₘ were 27.0 s⁻¹ and 1.04×10⁵ M⁻¹s⁻¹, indicating that nitrilase bll6402 from Bradyrhizobium japonicum USDA110 was very active for the hydrolysis of mandelonitrile to the corresponding carboxylic acid.

### Example 7

### Substrate Specificity

The specific activities of nitrilase bll6402 toward 24 different nitriles with structural diversity were measured by the quantification of the amount of ammonia released during the hydrolysis. A reaction mixture containing 20 or 40 μg of purified bll6402 enzyme, 25 mM substrate in potassium phosphate buffer (1 ml, 100 mM, pH 7.2) was incubated at 30° C. The conversion was determined by measuring the amount of ammonia produced in the reaction using Bertholet assay as described above.

For the convenience of comparison, the relative activities for these nitriles are presented in Table 2. The specific activity of phenylacetonitrile was 5.3 U/mg and its relative activity was defined as 100. Table 2 clearly shows that nitrilase bll6402 possessed highest activity for the hydrolysis of mandelonitrile (4.6 times that of phenylacetonitrile). All the other nitriles are much less active. This nitrilase was much less active towards other tested nitriles, including aromatic, aliphatic, and arylacetonitriles. Thus nitrilase bll6402 is indeed a mandelonitrile hydrolase, which effectively catalyzes the hydrolysis of mandelonitrile to mandelic acid.

The present observation is consistent with the prediction derived from the functional analysis of genetic organization of nitrilase gene bll6402 within the chromosome of Bradyrhizobium japonicum strain USDA110.

### Example 8

### Hydrolysis of Aromatic α-Hydroxy Nitriles to Carboxylic Acids

To further characterize the efficiency of nitrilase bll6402 to catalyze the hydrolysis of mandelonitrile derivatives to the corresponding carboxylic acids, the hydrolysis of several aromatic α-hydroxy nitriles were carried out and the product carboxylic acids were characterized by HPLC analysis. An example procedure was as follows: aromatic α-hydroxynitriles (25 mM) were treated with 0.16 mg nitrilase bll6402 in 1 mL of potassium phosphate buffer (100 mM, pH 7.2). The reaction mixture was incubated overnight at 30° C. A sample was withdrawn and analyzed by HPLC (column: Agilent C8, eluent: MeOH:H₂O=20:80, flow rate: 1 mL/min, detector: 254 nm) and the products were identified by comparison with the authentic standards.

The results are presented in Table 2 and show that mandelonitrile, α-hydroxy-α-(p-hydroxyphenyl)acetonitrile and α-hydroxy-α-(m-hydroxy-phenyl)acetonitrile were hydrolyzed to the corresponding carboxylic acids in high yields under the action of nitrilase bll6402. α-Trimethylsilyloxyphenylacetonitrile was converted to mandelic acid with concomitant cleavage of trimethylsilyl group. This may have been due to the instability of the trimethylsilyl-oxygen bond under aqueous conditions. Because α-trimethylsilyloxyphenylacetonitrile showed lower activity than mandelonitrile (Table 2), the cleavage of trimethylsilyl group likely precedes the hydrolysis of nitrile group and is the rate-determining step in the reaction sequence.

Thus, Bradyrhizobium japonicum strain USDA110 harbors a gene (bll6402) encoding a mandelonitrile hydrolase, which, as the present disclosure reveals, is an efficient catalyst for the hydrolysis of mandelonitrile derivatives to the corresponding aromatic α-hydroxy carboxylic acids.

### Example 9

### Comparison of bll6402 with Other Nitrilases

The alignment of amino acid sequences of various nitrilases from different bacteria by Clustal V of Lasergene program from DNAStar is shown in FIG. 2. Nitrilase bll6402 from B. japonicum USDA110 has highest degree of amino acid sequence identity (55.7%) with nitrilase from P. fluorescens EBC191 among all the nitrilases aligned. The nitrilase gene from P. fluorescens EBC191 was found to be physically connected to several genes on the mandelate pathway in the Pseudomonas fluorescens genome. Thus, it seems reasonable that the predicted cyanogenic glycoside metabolic pathway in Scheme 2 may exist in B. japonicum and a natural function of nitrilase bll6402 may be the detoxification and utilization of these hydroxynitriles produced in the metabolism of cyanogenic glycosides.

Two arylacetonitrilases from Alcaligenes faecalis ATCC8750 and JM3 hydrolyze mandelonotrile, but their activities are only 12-15% of those for phenylacetonitrile. The amino acid sequence of nitrilase from A. faecalis ATCC8750 is not available. Nitrilase bll6402 shows 43.1% sequence identity to the nitrilase from A. faecalis JM3 (accession No. D13419) (FIG. 2). Thus nitrilase bll6402 is distinct from these known phenylacetonitrilases as a mandelonitrile hydrolase although they all show activity toward mandelonitrile and phenylacetonitrile.

### Example 10

The GC analysis was performed on a Hewlett Packard 5890 series II plus gas chromatograph equipped with autosampler, EPC, split/splitless injector, FID detector and 25 m×0.25 mm CP-Chirasil-Dex CB capillary column. ¹H and ¹³C NMR spectra were recorded on a 400 MHz Bruker AVANCE DRX-400 Multinuclear NMR spectrometer. The substrate nitriles and acid standard samples were purchased from Sigma-Aldrich and used as received. The nitrilase enzyme was produced according to Examples 2-4.

### Example 11

### Effects of Temperature, pH and Organic Solvents

For the determination of temperature effect, a mixture of nitrilase (0.5 mg) with benzyl cyanide (50 mM) in 1.0 ml of potassium phosphate buffer (100 mM, pH 7.1) was incubated at 25° C., 30° C., 37° C. and 45° C., respectively. Aliquots (100 μl) were taken after 1, 2, 4, and 16 hrs intervals, and acidified with 10 μl of 1M HCl. The formed phenylacetic acid was extracted into 200 μl of tert-butyl methyl ether, dried over anhydrous sodium sulfate, and quantified by GC analysis after converting to methyl ester with diazomethane.

The pH effect was studied using the following buffer: sodium citrate buffer (pH 4.91 and 5.44), potassium phosphate buffer (pH 6.07, 6.52, 7.10, 7.58, 7.98 and 8.49) and sodium bicarbonate buffer (pH 8.98). A mixture of nitrilase (0.5 mg) with benzyl cyanide (50 mM) in 1.0 ml of the respective buffer (100 mM) was incubated at 30° C. Aliquots (100 μl) were taken after 1, 2, 4, and 20 hrs intervals, and acidified with 10 μl of 1M HCl. The formed phenylacetic acid was extracted into 200 μl of tert-butyl methyl ether, dried over anhydrous sodium sulfate, and quantified by GC analysis after converting to methyl ester with diazomethane.

The organic solvent effects were evaluated with dimethyl sulfoxide (DMSO), tert-butyl methyl ether, hexane, toluene and butyl acetate. A mixture of nitrilase (0.5 mg) with benzyl cyanide (50 mM) in potassium phosphate buffer (100 mM, pH 7.1) with indicated amount of organic solvent (total volume 1 ml) was incubated at 30° C. The reaction mixture was acidified with 100 μl of 1M HCl after 4 hours. The formed phenylacetic acid was extracted into 1 ml of tert-butyl methyl ether, dried over anhydrous sodium sulfate, and quantified by GC analysis after converting to methyl ester with diazomethane.

The kinetic parameters of nitrilase bll6402 for the hydrolysis of phenylacetonitrile were determined by Bertholet assay, and the Vₘₐₓ and Kₘ were 2.2 U/mg and 0.1 mM, respectively. The k_{cat} and k_{cat}/Kₘ were 1.36 s⁻¹ and 13600 s⁻¹M⁻¹, indicating nitrilase bll6402 from Bradyrhizobium japonicum strain USDA110 is very active for the hydrolysis of phenylacetonitrile to phenylacetic acid. Therefore, the dependences of the nitrilase activity on temperature and pH, and its tolerance of organic solvents were studied with phenylacetonitrile as the substrate.

The activities of nitrilase bll6402 toward phenylacetonitrile at different temperatures were studied by measuring the conversion of the substrate to phenylacetic acid at four time intervals, and the data are presented in FIG. 1. It can be seen that the reaction rate increased at higher temperature, and this nitrilase was active at 45° C. for at least 2 hours, indicating nitrilase bll6402 is thermal stable with the temperature optimum of 45° C. or higher. Results are shown in FIG. 3.

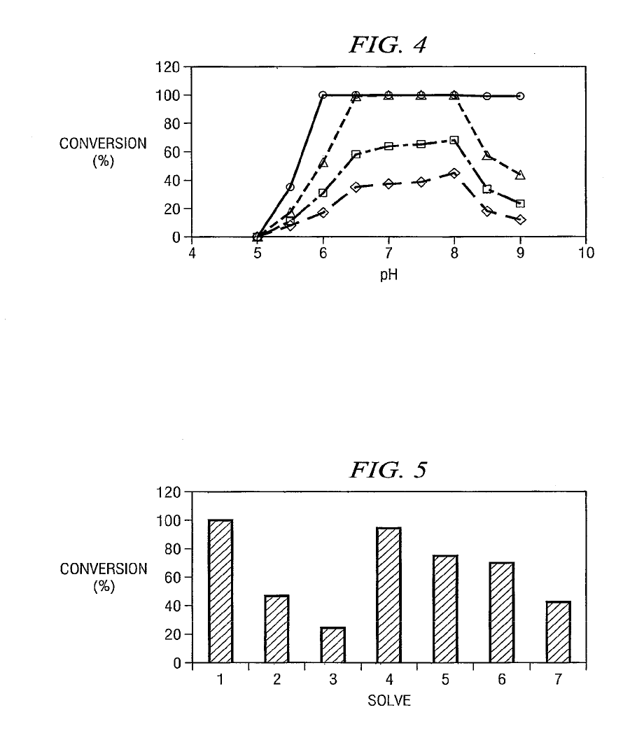

The activity of nitrilase bll6402 toward the hydrolysis of phenylacetonitrile was measured from pH 5 to 9 (FIG. 4). It has been found that the enzyme was active in this pH range with a broad pH optimum from pH 6.5 to 8. This suggests that nitrilase bll6402 possesses a broad working pH range.

The organic solvent tolerance of nitrilase bll6402 was investigated with phenylacetonitrile as the substrate. Several solvents were tested and the results are shown in FIG. 5. It is worth noting that this enzyme was active in the presence of all of five tested organic solvents, especially it retained more than 70% activity in the biphasic systems containing 30% (v/v) of hexane, tert-butyl methyl ether or toluene. This indicates that nitrilase bll6402 is a very robust enzyme in organic solvents. From the synthetic point of view, this property is particularly important and useful for the nitriles with low solubility in aqueous buffers.

### Example 12

### Hydrolysis of Various Nitriles

To a solution of substrate (0.5 mmole) in potassium phosphate buffer (5 ml, 100 mM, pH=7.2), enzyme (1 mg) was added and the reaction mixture was incubated at 30° C. for 14 hrs. The mixture was acidified with 1 N HCl solution to pH ˜5, saturated with NaCl and extracted with ethyl acetate. The organic extract was dried over anhydrous sodium sulfate. Removal of solvent provided the desired product, which was further purified by preparative TLC using ethyl acetate-hexane as eluting solvent. The product acids were characterized by gas chromatography or ¹H NMR analysis and compared the data with those of authentic samples. The GC analysis was performed after the acids were converted to methyl esters with freshly prepared diazomethane.

The above studies showed that nitrilase bll6402 was thermostable, had a wide working pH range and tolerance of organic solvents. These are important properties from the synthetic point of view. To further explore the synthetic applicability, the activity of this nitrilase to hydrolyze the various nitriles to the corresponding carboxylic acids was investigated. The substrate nitriles were treated with the enzyme in potassium phosphate buffer at 30° C. for 14 hrs. The products were isolated via preparative TLC and the yields were summarized in Table 1. The acid products were analyzed by gas chromatography or ¹H NMR and identified by comparing the data with those of the standard samples.

Nitrilase bll6402 efficiently hydrolyzed α-substituted phenylacetonitriles such as mandelonitrile and 2-phenylpropionitrile to the corresponding carboxylic acids. However, further increasing the size of the α-substituent (e.g. ethyl group) inhibited the activity. In addition to catalyzing the hydrolysis of hydrocinnamonitrile and 4-phenylbutyronitrile to the acids in high yields, this nitrilase also effectively hydrolyzed aliphatic nitriles without phenyl at the other end to the corresponding carboxylic acids. As shown in Table 4, the hydrolyses of benzonitrile and crotonitrile were much slower, indicating nitrilase bll6402 showed clear preference for aliphatic nitriles over aromatic and vinyl nitriles.

### Example 13

### Preparation of 1-cyanocycloalkaneacetonitriles—Selective Hydrolysis of Dinitriles

1-Cyanocycloalkaneacetonitriles were prepared by a modified procedure of the reported methods (using 1-cyanocyclokexaneacetonitrile as the example): Ethyl cyano acetate (1.0 g, 8.84 mmol) and cyclohexanone (7.10 mmol) were dissolved in anhydrous benzene (10 ml) containing ammonium acetate (1.77 mmol) and glacial acetic acid (0.95 ml). The reaction mixture was refluxed vigorously for 4 hrs, and the water formed during the reaction was removed with a Dean-Stark condenser placed under the refluxing condenser. Evaporation of the solvent afforded crude product, which was purified by column chromatography using ethyl acetate/hexane (10/90, v/v) as eluent. The α,β-unsaturated α-cyanoacetate was isolated as colorless oil in 95% yield. A solution of α,β-unsaturated α-cyanoacetate (5 mmol) and NaCN (8.5 mmol) in 90% ethanol (15 mL) was refluxed for 5 hrs. The resulting dark solution was evaporated, and residue was suspended in water (50 ml) and extracted with dichloromethane. The organic extract was dried over anhydrous Na₂SO₄. Removal of solvents afforded 1-cyanocyclokexaneacetonitrile as white solid (0.52 g, 70% yield).

Because of the synthetic importance of selective hydrolysis of dinitriles to carboxylic acids, nitrilase bll6402 was studied toward the hydrolysis of a series of dinitriles (e.g., scheme 6).

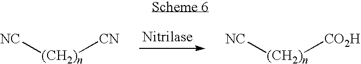

The results are presented in Table 5. As shown in Table 5, nitrilase bll6402 efficiently catalyzed the selective hydrolysis of α,ω-dinitriles to exclusively afford cyanocarboxylic acids. The selectivity was not dependent on the chain length. This was different from the results obtained with nitrilases from Arabidopsis thaliana, acidovorax facilis 72W and cyanobacterium Synechocystis sp. strain PCC 6803, in which di-acids were obtained as the chain length increased.

### Example 14

### Preparation of 1-cyanocycloalkaneacetic Acids

The general procedure was followed: 1-Cyanocycloalkaneacetonitiles (200 mg) was treated with the nitrilase (10 mg) in potassium phosphate buffer (50 ml, 100 mM, pH=7.2). The reaction mixture was incubated at 30° C. and monitored by ¹³C NMR. When dinitrile was consumed, the mixture was acidified with 1 N HCl solution to pH ˜5, saturated with NaCl and extracted with ethyl acetate. The organic extract was dried over anhydrous sodium sulfate. Removal of solvent provided the desired product, which was further purified by preparative TLC using ethyl acetate-hexane (35/65, v/v) as eluting solvent.

1-cyanocyclopentaneacetic acid: 210 mg, 92% yield; ¹H NMR (400 MHz, CDCl₃) δ=2.74 (s, 2H), 2.32-2.34″ (m, 2H), 1.91-1.93 (m, 2H), 1.7-1.85 (m, 4H); ¹³C NMR (100.6 MHz, CDCl₃) δ=175.6, 124.4, 42.3, 39.9, 38.6, 24.4.

1-cyanocyclohexaneacetic acid: 198 mg, 88% yield; ¹H NMR (400 MHz, CDCl₃) δ=2.64 (s, 2H), 2.13-2.16 (m, 2H), 1.64-1.78 (m, 5H), 1.35-1.41 (m, 2H), 1.20-1.27 (m, 1H); ¹³C NMR (100.6 MHz, CDCl₃) δ=175.1, 122.6, 42.2, 36.6, 35.7, 25.4, 23.1.

1-cyanocycloheptaneacetic acid: 120 mg, 54% yield; ¹H NMR (400 MHz, CDCl₃) δ=2.67 (s, 2H), 2.14-2.17 (m, 2H), 1.70-1.80 (m, 8H), 1.54-1.65 (m, 2H); ¹³C NMR (100.6 MHz, CDCl₃) δ=175.3, 123.6, 44.6, 39.2, 38.2, 28.1, 23.6.

Since 1-cyanocycloalkaneacetic acids were useful precursors for the synthesis of gabapentin and its analogs, nitrilase bll6402 was used for the preparation of 1-cyanocycloalkaneacetic acids such as 1-cyanocyclopentaneacetic acid, 1-cyanocyclohexaneacetic acid and 1-cyanocycloheptaneacetic acid as shown in Scheme 7.

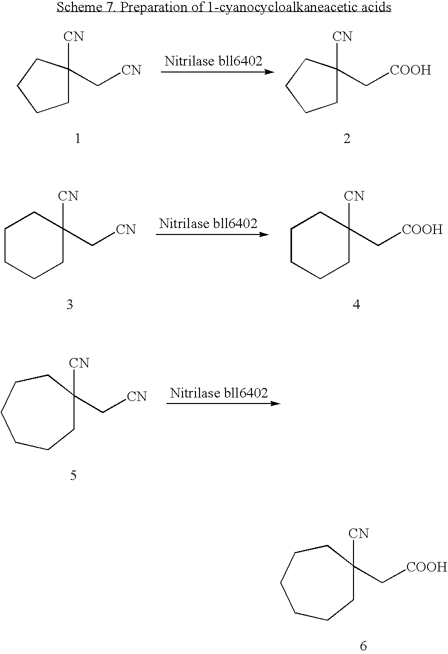

1-Cyanocycloalkaneacetonitiles were prepared by a modified procedure from literatures (Scheme 8). When 1-cyanocyclopentaneacetonitrile, 1-cyanocyclohexaneacetonitrile and 1-cyanocycloheptaneacetonitrile were treated with the nitrilase in potassium phosphate buffer, the corresponding cyanocarboxylic acids were isolated in 92, 88 and 54% yields, respectively, demonstrating that nitrilase bll6402 was a useful catalyst for the preparation of the precursors of gabapentin and its analogs via regioselective hydrolysis of 1-cyanocycloalkaneacetonitiles.

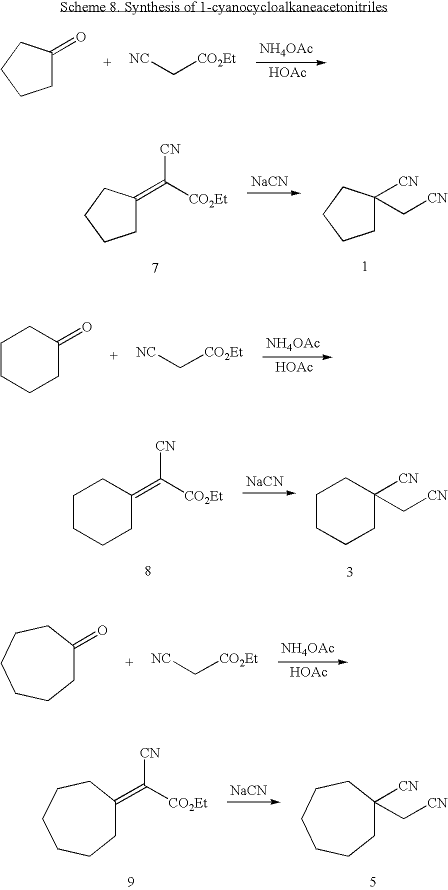

### Example 15

### Hydrolysis of Aromatic β-Hydroxy Nitriles to Carboxylic Acids

Nitrilase bll6402 was tested to determine whether it could also decompose β-hydroxy nitriles (e.g., Scheme 9).

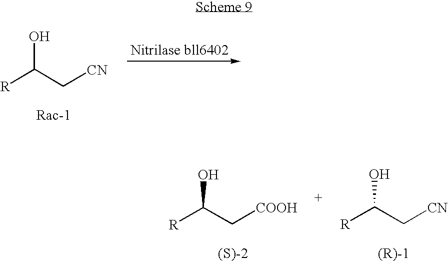

In the present example, β- and α-hydroxy nitriles are referenced by compound numbers according to formula I

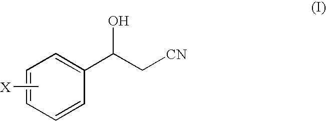

wherein in X=

1a 4-H

1b 4-F

1c 4-Cl

1d 4-CH₃

1e 4-OCH₃

1f 2-OCH₃

1g 3-OCH₃

1h 2-Cl

1i 2,4-Cl₂

or

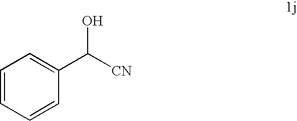

β-Hydroxy nitriles were prepared by the cyanization of α-bromoketones with sodium cyanide followed by NaBH₄ reduction. The obtained β-hydroxy nitriles were treated with purified nitrilase bll6402 in potassium phosphate buffer (100 mM, pH 7.0), and the reaction mixture was incubated overnight at 30° C. The mixture was then saturated with NaCl and extracted with ethyl acetate. The extract was dried over sodium sulfate, and evaporation of the solvent under reduced pressure afforded the crude products. The β-hydroxy carboxylic acids were separated from unreacted β-hydroxy nitriles by preparative thin-layer chromatography.

The ee values of both the product acids and the recovered nitriles were measured by chiral HPLC analysis, and their absolute configurations were determined by comparing the sign of optical rotation with the literature data. The enantiomeric ratios (E) were calculated using the equations proposed by Sih et al. (J. Am. Chem. Soc. 1982, 104, 7294-7299). The results are presented in Table 6 together with the data for the hydrolysis of mandelonitrile under the same conditions.

Nitrilase bll6402 catalyzed the enantioselective hydrolysis of aromatic β-hydroxy nitriles to give (S)-enriched β-hydroxy carboxylic acids with recovery of (R)-enriched β-hydroxy nitriles. The substituent on the benzene ring of β-hydroxy nitriles did not significantly affect the enzyme activity but exerted some effect on the enantioselectivity. Among the para-substituted aryl β-hydroxy nitriles, the hydrolysis of the substrate with the para-methoxy group (1e) showed the highest enantioselectivity with E being 43. The position of the substituent on the benzene ring also affected the enantioselectivity. For example, the enantiomeric ratios E for the substrates with a para- and meta-methoxy substituent were 43 (1e) and 52 (1g), respectively, whereas that of their counterpart with an ortho-methoxy group (1f) was only 5. In contrast, this nitrilase showed higher enantioselectivity for β-hydroxy nitrile with an ortho-chloro group (1h) than the one with a para-chloro substituent (1c). Thus, the enantioselectivity of this nitrilase was influenced by both the steric and electronic factors of the substituents on the benzene ring.

To test the role that the β-hydroxy group plays in the enantioselective hydrolysis of β-hydroxy nitrites, 3-phenylbutyronitrile (in which the hydroxy group was replaced with a methyl group) was treated with nitrilase bll6402. However, 3-phenylbutyronitrile was not hydrolyzed by nitrilase bll6402 under the same conditions. This indicates that the hydroxy group of β-hydroxy nitriles not only promotes the stereo-discrimination at the β-position but also plays a critical role in determining the enzyme activity.

### Example 16

### Codon Optimization of Nitrilase bll6402

An artificial DNA sequence encoding nitrilase bll6402 was prepared by reverse translation of the amino acid sequence of SEQ ID NO: 2 using DNAStar Lasergene99 (DNAStar, Inc. Madison, Wis.). The resulting sequence, SEQ ID NO: 5, is codon optimized for expression in E. coli.

TABLE 1
Organisms and Locus tags of putative nitrilase genes

| Organism | Putative nitrilase | Locus tag |
|---|---|---|
| Bordetella bronchiseptica | NP_887662^{a}, 310 a.a | BB1116 |
| RB50 | | |
| Bradyrhizobium japonicum | NP_770037, 321 a.a | blr3397 |
| USDA110 | NP_773042, 334 a.a | bll6402 |
| Klebsiella pneumoniae | NP_943299, 334 a.a | LV044 |
| Neisseria gonorrhoeae | YP_208564, 304 a.a | NGO1514 |
| FA1090 | | |
| Nocardia farcinica IFM | YP_119480, 336 a.a | nfa32690 |
| 10152 | | |
| Photorhabdus luminescens | NP_928542, 335 a.a | plu1231 |
| subsp. Laumondii TTO1 | | |
| Pseudomonas syringae pv. | NP_790047, 347 a.a | PSPTO0189 |
| tomato str. DC3000 | | |
| Ralstonia solanacearum | NP_519944, 343 a.a | RSc1823 |
| GMI1000 | | |
| Rhodopseudomonas palustris | NP_946909, 349 a.a | RPA1563 |
| CGA009 | NP_949502, 317 a.a | RPA4166 |
| Silicibacterpomeroyi DSS-3 | YP_164946, 344 a.a | SPOA0114 |
| Synechococcus elongates | YP_171411, 334 a.a | syc0701_d |
| PCC6301 | | |
| Synechocystis sp. PCC 6803 | NP_442646, 346 a.a | sll0784 |
| Synechococcus sp. WH8102 | NP_897518, 338 a.a | SYNW1425 |
| Zymomonas mobilis subsp. | YP_162942, 329 a.a | ZMO1207 |
| Mobilis ZM4 | | |

^{a}Accession number of putative nitrilases.

TABLE 2
The relative activity of nitrilase bll6402 from Bradyrhizobium japonicum
USDA110 with various nitrile substrates

| | Relative |
|---|---|
| Substrate | Activity^{a} |
| Mandelonitrile | 460 |
| α-Trimethylsilyloxyphenylacetonitrile | 165 |
| 2-Phenylglycinonitrile | 3 |
| α-N,N- | 1 |
| Dimethylaminophenylacetonitrile | |
| α-phenylpropionitrile | 10 |
| α-Phenylbutyronitrile | 8 |
| Phenylacetonitrile | 100 |
| Hydrocinnamonitrile | 13 |
| 4-Phenylbutyronitrile | 4 |
| 3-Indolylacetonitrile | 2 |
| Benzonitrile | 1 |
| 4-Acetylbenzonitrile | 2 |
| n-Butyronitrile | 3 |
| 4-Chlorobutyronitrile | 6 |
| Valeronitrile | 6 |
| Hexanenitrile | 16 |
| Heptanenitrile | 22 |
| Crotononitrile | 3 |
| Allyl cyanide | 2 |
| 1,4-Dicyanobutane | 5 |
| 2-Methylglutaronitrile | 3 |
| 3-Aminopropionitrile | 1 |
| 3-Hydroxypropionitrile | 1 |
| Methylthioacetonitrile | 1 |

^{a}The specific activity of phenylacetonitrile was 5.3 U/mg and its relative activity was defined as 100.

TABLE 3
Hydrolysis of aromatic α-hydroxynitriles

| Substrate | Product | Conversion (%)^{a} |
|---|---|---|
| Mandelonitrile | Mandelic acid | 98 |
| α-Trimethylsilyloxy- | Mandelic acid^{b} | 99 |
| phenylacetonitrile | | |
| α-Hydroxy-α-(p- | α-Hydroxy-α-(p- | 88 |
| hydroxyphenyl)acetonitrile | hydroxyphenyl)acetic | |
| | acid | |
| α-Hydroxy-α-(m- | α-Hydroxy-α-(m- | 94 |
| hydroxyphenyl)acetonitrile | hydroxyphenyl)acetic | |
| | acid | |

^{a}The conversion was determined by HPLC analysis.
^{b}Trimethylsilyl group was cleaved under the reaction conditions.

TABLE 4
Hydrolysis of various nitriles catalyzed by the nitrilase
from Bradyrhizobium japonicum USDA110

| | | Yield |
|---|---|---|
| Substrate | Product | (%)^{[a]} |
| Mandelonitrile | Mandelic acid | 89 |
| 2-Trimethylsilyloxy-2- | Mandelic acid | 91 |
| phenylacetonitrile | | |
| 2-phenylpropionitrile | 2-phenylpropionic acid | 88 |
| 2-Phenylbutyronitrile | | 0 |
| Phenylacetonitrile | Phenylacetic acid | 95 |
| Hydrocinnamonitrile | Hydrocinnamic acid | 82 |
| 4-Phenylbutyronitrile | 4-Phenylbutyric acid | 92 |
| Benzonitrile | Benzoic acid | 7 |
| n-Butyronitrile | Butyric acid | 61 |
| Valeronitrile | Valeronic acid | 81 |
| Hexanenitrile | Hexanoic acid | 72 |
| Heptanenitrile | Heptanoic acid | 79 |
| Crotonitrile | Crotonic acid | 25 |
| Allyl cyanide | 3-Butenoic acid | 87 |
| Methylthioacetonitrile | Methylthioacetic acid | 96 |

^{[a]}Isolated yield, the products were identified by gas chromatography or ¹H NMR analysis.

TABLE 5
Selective hydrolysis of dinitriles catalyzed by nitrilase bll6402

| Dinitrile | Yield (%) |
|---|---|
| α,α-Dimethylmalononitrile | 93 |
| Fumaronitrile | 84 |
| Succinonitrile | 97 |
| Glutaronitrile | 93 |
| 1,4-Dicyanobutane | 97 |
| 1,5-Dicyanopentane | 90 |
| 1,6-Dicyanohexane | 88 |
| Sebeconitrile | 88 |

TABLE 6
Enantioselective Hydrolysis of β-Hydroxy
Nitriles Catalyzed by Nitrilase bll6402

| | |
|---|---|
| recovered | product acid |
| nitrile (R)-1 | (S)-2 |

| | β-hydroxy | yield | ee | yield | ee | |
|---|---|---|---|---|---|---|
| entry | nitrile | (%)^{a} | (%) | (%)^{a} | (%) | E^{b} |
| 1 | la | 41 | 53 | 36 | 48 | 5 |
| 2 | lb | 37 | 74 | 38 | 60 | 9 |
| 3 | lc | 40 | 53 | 32 | 65 | 8 |
| 4 | ld | 35 | 76 | 40 | 42 | 5 |
| 5 | le | 57 | 66 | 27 | 90 | 43 |
| 6 | if | 46 | 75 | 36 | 43 | 5 |
| 7 | lg | 46 | 67 | 35 | 91 | 52 |
| 8 | lh | 40 | 75 | 32 | 84 | 27 |
| 9 | li | 42 | 37 | 39 | 59 | 13 |
| 10 | lj | 0 | —^{c} | 98 | 0 | —^{c} |

^{a}Isolated yield.
^{b}The enantiomeric ratio is calculated by the equation E = ln[(l − c)(1 − ee(S))]/ln[(1 − c)(1 + ee(S))], where c is calculated by the equation c = [ee(S) + eeₒ]/[ee(S) + ee(P)], ee(S) is the ee of the substrate, eeₒ is the initial ee of the substrate, and ee(P) is the ee of the product.
^{c}Not applicable.

As will be understood by those of ordinary skill in the art, other equivalent or alternative methods of rational genome mining can be envisioned without departing from the essential characteristics thereof. In addition, other equivalent or alternative compositions and methods of converting nitriles to carboxylic acids can be envisioned without departing from the essential characteristics thereof.

For example, methods of the disclosure may be practiced on a laboratory scale or on a pharmaceutical manufacturing or other industrial scale. Similarly, methods may adapted to being practiced on a micro or nano scale as desired. Reaction conditions (e.g., temperature, pH, solvents, buffers) may be modified as required or desired. Also, the disclosure is not limited to any particular chemical pathway or mechanism of action. All or part of a system of the disclosure may be configured to be disposable and/or reusable. From time to time, it may be desirable to clean, repair, and/or refurbish a reusable component. Moreover, one of ordinary skill in the art will appreciate that a feature and/or advantage of one embodiment may be associated with some or all other embodiments. Likewise, a limitation of one embodiment may not be associated with some or all other embodiments.

These equivalents and alternatives along with apparent changes and modifications are intended to be included within the scope of the present disclosure. Accordingly, the foregoing description is intended to be illustrative, but not limiting, of the scope of the disclosure as illustrated by the appended claims.

1-46. (canceled)

47. A system operable to convert a nitrile to a carboxylic acid comprising:
an isolated or a purified nitrilase, wherein the nitrilase:
comprises a polypeptide having an amino acid sequence that comprises the sequence of SEQ ID NO: 2, and
has a catalytic activity sufficient to convert, in a solvent at a pH between about 5 and about 9 and a temperature of less than about 50° C., at least about 50% of a nitrile to a carboxylic acid;
a nitrile selected from a group consisting of an α-hydroxy nitrile, a β-hydroxy nitrile, or a dinitrile;
a means for contacting the nitrilase with the nitrile; and
conditions for catalysis of the nitrile into the carboxylic acid.

48. The system of claim 47, wherein the conversion of the dinitrile to the carboxylic acid comprises hydrolysis of at least one nitrile group of the dinitrile.

49. The system of claim 47, wherein the conversion of the dinitrile comprises a selective hydrolysis of one nitrile group of the dinitrile.

50. The system of claim 49, wherein the selective hydrolysis is a regioselective hydrolysis.

51. The system of claim 49, wherein the selective hydrolysis of one nitrile group of the dinitrile produces a cyano-carboxylic acid.

52. The system of claim 51, wherein the cyano-carboxylic acid is a precursor for a drug with anti-depressant properties, a precursor for a drug with anti-convulsant properties or a precursor for an γ-amino butyric acid (GABA) analog.

53. The system of claim 52, wherein the cyano-carboxylic acid is a 1-cyanocycloalkaneacetic acid or an analog thereof.

54. The system of claim 53, wherein the 1-cyanocycloalkaneacetic acid is 1-cyanocyclohexaneacetic acid, 1-cyanocyclopentaneacetic acid, 1-cyanocycloheptaneacetic acid, or analogs thereof.

55. The system of claim 52, further operable to convert the cyano-carboxylic acid into gabapentin, pregablin or analogs thereof, the system further comprising:
conditions for reducing the cyano group of the cyano-carboxylic acid or analog thereof to an amino group.

56. The system of claim 47, wherein the isolated or purified nitrilase further enantioselectively catalyzes the conversion of a beta-hydroxynitrile to a S-beta-hydroxy carboxylic acid.

57. An isolated or purified nitrilase enzyme wherein the nitrilase:
comprises a polypeptide having an amino acid sequence that comprises the sequence of SEQ ID NO: 2;
has a catalytic activity sufficient to convert, in a solvent at a pH between about 5 and about 9 and a temperature of less than about 50° C., at least about 50% of a nitrile to a carboxylic acid; and
catalyzes the selective hydrolysis of a dinitrile to a cyano-carboxylic acid or enantioselectively catalyzes the conversion of a beta-hydroxynitrile to a S-beta-hydroxy carboxylic acid.

58. The isolated or purified nitrilase of claim 57, wherein the nitrile is an α-hydroxy nitrile, a β-hydroxy nitrile, or a dinitrile.

59. The isolated or purified nitrilase of claim 57, wherein the dinitrile is an α,ω-dinitrile.

60. The isolated or purified nitrilase of claim 59, wherein the carboxylic acid is a cyano-carboxylic acid.

61. The isolated or purified nitrilase of claim 60, wherein the cyano-carboxylic acid is a precursor of a drug with anti-convulsant properties or anti-depressant properties.

62. The isolated or purified nitrilase of claims 57, wherein the selective hydrolysis of the dinitrile is independent of chain length.

63. The isolated or purified nitrilase of claim 57, wherein the dinitrile is α,α-Dimethylmalononitrile, Fumaronitrile, Succinonitrile, Glutaronitrile, 1,4-Dicyanobutane, 1,5-Dicyanopentane, 1,6-Dicyanohexane, or Sebeconitrile.

64. The isolated or purified nitrilase of claim 57, wherein the β-hydroxy nitrile has the formula: 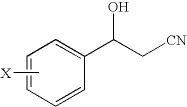
wherein, X=
1a 4-H
1b 4-F
1c 4-Cl
1d 4-CH₃
1e 4-OCH₃
1f 2-OCH₃
1g 3-OCH₃
1h 2-Cl
1i 2,4-Cl₂
or

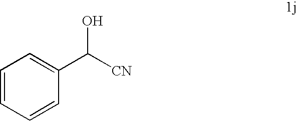

65. The isolated or purified nitrilase of claim 64, wherein the nitrilase has higher enantioselectivity to an ortho-chloro group (1h) of the β-hydroxy nitrile as compared to the entioselectivity to a para-chloro group (1c) of the β-hydroxy nitrile.

66. The isolated or purified nitrilase of claim 57, wherein the solvent is an aqueous solvent.

67. The isolated or purified nitrilase of claim 57, wherein the solvent is a biphasic solvent comprising water and an organic solvent up to about 30% (V/V).

68. The isolated or purified nitrilase of claim 57, wherein the organic solvent is selected from the group consisting of dimethyl sulfoxide, tert-butyl methyl ether, hexane, toluene, butyl acetate, and combinations thereof.

69. A method of converting a dinitrile to an ω-cyanocarboxylic acid comprising:
contacting an α,ω-dinitrile with an isolated or a purified nitrilase, wherein said nitrilase:
comprises a polypeptide having an amino acid sequence that comprises the sequence of SEQ ID NO: 2,
has a catalytic activity sufficient to convert, in a solvent at a pH between about 5 and about 9 and a temperature of less than about 50° C., at least about 50% of a nitrile to a carboxylic acid; and
catalyzes the selective hydrolysis of a dinitrile to a cyano-carboxylic acid;
whereby selective hydrolysis of the α,ω-dinitrile by the nitrilase produces an ω-cyanocarboxylic acid.

70. The method of claim 69, wherein the ω-cyanocarboxylic acid is further converted to a GABA analog, a drug with anti-convulsant properties or a drug with anti-depressant properties by reducing the cyano group of the cyano-carboxylic acid to an amino group.

71. The method of claim 70, wherein the ω-cyanocarboxylic acid is a 1-cyanocycloalkaneacetic acid or an analog thereof.

72. The method of claim 71, wherein the 1-cyanocycloalkaneacetic acid is 1-cyanocyclohexaneacetic acid, 1-cyanocyclopentaneacetic acid, 1-cyanocycloheptaneacetic acid, or an analog thereof.

73. The method of claim 72, wherein the 1-cyanocyclohexaneacetic acid is further converted to Gabapentin or an analog thereof.

74. The method of claim 70, wherein the ω-cyanocarboxylic acid is further converted to Pregablin or an analog thereof.

75. A method of converting a nitrile to a carboxylic acid, said method comprising:
contacting a nitrile with a nitrilase under conditions that permit hydrolysis, wherein said nitrilase comprises:
an isolated or purified polypeptide having an amino acid sequence that is more than about 95% identical to SEQ ID NO: 2; and
having a catalytic activity sufficient to convert, in a solvent at a pH between about 5 and about 9 and a temperature of less than about 50° C., at least about 50% of a nitrile to a carboxylic acid.

76. A method according to claim 75, wherein the nitrile is selected from the group consisting of an α-hydroxy nitrile, a β-hydroxy nitrile, and an α,ω-dinitrile.

77. A method according to claim 76, wherein the α-hydroxy nitrile comprises an aromatic α-hydroxy nitrile.

78. A method according to claim 75, wherein the nitrile is selected from the group consisting of mandelonitrile, α-trimethylsilyloxyphenylacetonitrile, 2-phenylglycinonitrile, α-n,n-dimethylaminophenylacetonitrile, α-phenylpropionitrile, α-phenylbutyronitrile, phenylacetonitrile, hydrocinnamonitrile, 4-phenylbutyronitrile, 3-indolylacetonitrile, benzonitrile, 4-acetylbenzonitrile, n-butyronitrile, 4-chlorobutyronitrile, valeronitrile, hexanenitrile, heptanenitrile, crotononitrile, allyl cyanide, 1,4-dicyanobutane, 2-methylglutaronitrile, 3-aminopropionitrile, 3-hydroxypropionitrile, and methylthioacetonitrile.

79. A method according to claim 75, wherein the nitrile is selected from the group consisting of 2-trimethylsilyloxy-2-phenylacetonitrile, 2-phenylpropionitrile, 2-phenylbutyronitrile, phenylacetonitrile, hydrocinnamonitrile, 4-phenylbutyronitrile, benzonitrile, n-butyronitrile, valeronitrile, hexanenitrile, heptanenitrile, crotonitrile, allyl cyanide, and methylthioacetonitrile.

80. A method according to claim 75, wherein the carboxylic acid is selected from the group consisting of an α-hydroxycarboxylic acid, a β-hydroxycarboxylic acid, and an ω-cyanocarboxylic acid.

81. A method according to claim 75, wherein the carboxylic acid is selected from the group consisting of mandelic acid, α-hydroxy-α-(p-hydroxyphenyl)acetic acid, α-hydroxy-α-(m-hydroxyphenyl)acetic acid, 2-phenylpropionic acid, phenylacetic acid, hydrocinnamic acid, 4-phenylbutyric acid, benzoic acid, butyric acid, valeronic acid, hexanoic acid, heptanoic acid, crotonic acid, 3-butenoic acid, and methylthioacetic acid.

82. A method of converting a β-hydroxy nitrile to a corresponding β-hydroxycarboxylic acid, said method comprising:
contacting a β-hydroxy nitrile with a nitrilase under conditions that permit conversion of the β-hydroxy nitrile to the corresponding β-hydroxycarboxylic acid, wherein said nitrilase comprises:
an isolated or purified polypeptide having an amino acid sequence that is more than about 95% identical to SEQ ID NO: 2;
and having a catalytic activity sufficient to convert, in a solvent at a pH between about 5 and about 9 and a temperature of less than about 50° C., at least about 50% of a nitrile to a carboxylic acid.

83. A method of converting an α,ω-dinitrile to a corresponding ω-cyanocarboxylic acid, said method comprising:
contacting an α,ω-dinitrile with a nitrilase under conditions that permit conversion of the α,ω-dinitrile to the corresponding ω-cyanocarboxylic acid,
wherein said nitrilase comprises an isolated or purified polypeptide having an amino acid sequence that is more than about 95% identical to SEQ ID NO: 2 and having a catalytic activity sufficient to convert, in a solvent at a pH between about 5 and about 9 and a temperature of less than about 50° C., at least about 50% of a nitrile to a carboxylic acid.

84. A method according to claim 83, wherein the catalytic activity of the nitrilase for conversion of the α,ω-dinitrile to the corresponding ω-cyanocarboxylic acid is independent of chain length.
